(19) United States
(12) Patent Application Publication
(10) Pub. No.: US 2019/0053786 A1
(43) Pub. Date: Feb. 21, 2019

(54) DELAYED EXCITATION ULTRASONIC IMAGING METHOD AND APPARATUS AND DELAYED EXCITATION SYSTEM

(71) Applicant: Shenzhen Institutes of Advanced Technology, Shenzhen (CN)

(72) Inventors: Weibao QIU, Shenzhen (CN); Peitian MU, Shenzhen (CN); Hairong ZHENG, Shenzhen (CN)

(21) Appl. No.: 16/167,060

(22) Filed: Oct. 22, 2018

Related U.S. Application Data
(63) Continuation of application No. PCT/CN2016/083132, filed on May 24, 2016.

(30) Foreign Application Priority Data
Apr. 22, 2016 (CN) 201610257356.2

(51) Int. Cl. A61B 8/00; A61B 8/08

(52) U.S. Cl. CPC A61B 8/4483; A61B 8/56; A61B 8/5215; A61B 8/5207

(57) ABSTRACT

A delayed excitation ultrasonic imaging method includes: generating a regulating clock, and performing, according to the regulating clock, delayed excitation on an ultrasonic transducer once or multiple times; performing data collection on an ultrasonic echo signal of the ultrasonic transducer and signals after each time of performing delayed excitation; synthesizing and superimposing data collected to obtain high sampling rate data; and performing ultrasonic imaging according to the high sampling rate data. A delayed excitation ultrasonic imaging apparatus and system are also provided. By performing delayed excitation on the ultrasonic transducer, the high-frequency ultrasonic imaging is realized by low-frequency sampling and delayed excitation, which can greatly reduce the cost of the ultrasonic imaging system.

### CROSS-REFERENCE TO RELATED APPLICATIONS

This application is a continuation of International Application No. PCT/CN2016/083132, filed on May 24, 2016, which claims priority to Chinese Patent Application No. 201610257356.2, and filed on Apr. 22, 2016, both of which are hereby incorporated by reference in their entireties.

### TECHNICAL FIELD

The present invention relates to ultrasonic imaging technical field, in particular to a delayed excitation ultrasonic imaging method and apparatus, and a delayed excitation system.

### BACKGROUND

Medical imaging generally refers to a technology and a processing process of obtaining an image of interior tissues of a human body or a certain part of the human body in a non-invasive manner for the purpose of medical treatment or medical research, which is inference calculus of an inverse problem, i.e., causes (characteristics of living tissue) are inversely inferred from consequences (an observed image signal). Medical imaging is used very widely in clinic, provides a great scientific and intuitive basis for diagnosis of diseases, can better cooperate with clinical symptoms, laboratory tests and etc., and plays an irreplaceable role in the final accurate diagnosis of the diseases.

Medical imaging refers to in general the course of checking parts of a human body that cannot be examined by nonsurgical means by modern imaging technologies such as X-ray imaging, X-ray computed tomography (CT), magnetic resonance imaging (MRI), ultrasonic imaging (US), Optical coherence tomography (OCT), and the like. Compared with other imaging technologies, medical ultrasonic imaging has unique advantages such as good real-time, no damage, no pain, high imaging accuracy and a low system cost, etc., and currently has been widely used in clinical medical detection.

An ultrasonic transducer is an apparatus that can convert an electric signal and an acoustic signal. Due to the piezoelectric effect of a material, after having received the electric signal, the ultrasonic transducer can convert the electric signal into mechanical vibration and emit ultrasonic waves; or, after having received the ultrasonic wave, the ultrasonic transducer can convert the mechanical vibration into the electric signal, and in terms of use function, most of the ultrasonic transducers are capable of both receiving and transmitting. In an ultrasonic signal receiving link, an acoustic signal is converted into an electric signal. In an ultrasonic signal transmitting link, an electric signal is converted into an acoustic signal. For conventional ultrasonic imaging, generally, the working frequency of the ultrasonic transducer is below 5 MHz. For high-frequency ultrasonic imaging, generally, the working frequency of the ultrasonic transducer is above 10 MHz.

An analog-to-digital conversion chip is an integrated chip that converts an analog signal into a digital signal. Based on the need of satisfying Nyquist sampling theorem, a sampling rate of the analog-to-digital conversion chip should be at least twice or more the frequency of the measured signal, and should preferably be five times or higher if better sampling data is desired. For a 40 MHz ultrasonic transducer that is currently used more, it has been very difficult for a conventional analog-to-digital conversion chip to obtain better sampling data (the frequency numerical values involved in the present invention, such as 40 MHz, 80 MHz, and the like, are used only as examples to facilitate descriptions, and the solutions of the present invention are not merely limited to these frequency values).

In a conventional design solution, the 40 MHz ultrasonic transducers currently used more need to use high-speed analog-to-digital conversion chips of 200 MHz or higher to obtain better sampling data, but the high-speed analog-to-digital conversion chips of 200 MHz not only has a very high price but also is often limited to China, so the cost of the ultrasonic imaging system is very high.

For the problem of a high cost of the ultrasonic imaging system in the related art, an effective solution has not yet been proposed.

### SUMMARY

The present invention provides a delayed excitation ultrasonic imaging method and apparatus, and a delayed excitation system, to at least solve the problem of a high cost of the ultrasonic imaging system in the related art.

According to one aspect of the present invention, there is provided with a delayed excitation ultrasonic imaging method, comprising: generating a regulating clock, and performing, according to the regulating clock, delayed excitation on an ultrasonic transducer once or multiple times; performing data collection on an ultrasonic echo signal of the ultrasonic transducer and signals after each time of performing delayed excitation; synthesizing and superimposing data as collected to obtain high sampling rate data; and performing ultrasonic imaging according to the high sampling rate data.

Preferably, the step of performing delayed excitation on the ultrasonic transducer once according to the regulating clock includes: taking one period of the regulating clock as a delay period, and performing delayed excitation on the ultrasonic transducer once according to the delay period.

Preferably, the step of performing delayed excitation on the ultrasonic transducer multiple times according to the regulating clock includes: taking one period of the regulating clock as a delay period, and performing delayed excitation on the ultrasonic transducer once according to this delay period; taking two periods of the regulating clock as a delay period, and performing delayed excitation on the ultrasonic transducer once according to this delay period; in a similar fashion, taking multiple periods of the regulating clock as a delay period, and performing delayed excitation on the ultrasonic transducer once according to this delay period.

Preferably, the step of performing data collection on the ultrasonic echo signal of the ultrasonic transducer and the signals after each time of performing delayed excitation includes: performing data collection on the ultrasonic echo signal at a rising edge of a sampling clock; and performing data collection on the signals after each time of performing delayed excitation at a rising edge of the sampling clock.

Preferably, the step of synthesizing and superimposing the data as collected to obtain the high sampling rate data includes: synthesizing and superimposing the data as collected in accordance with a time-sequential relationship of delayed excitation to obtain the high sampling rate data.

Preferably, the ultrasonic transducer is a single ultrasonic transducer, or an ultrasonic array transducer consisting of a plurality of ultrasonic transducer elements.

According to another aspect of the present invention, there is provided with a delayed excitation ultrasonic imaging apparatus, wherein the delayed excitation ultrasonic imaging apparatus includes: a delayed excitation module configured to generate a regulating clock, and perform, according to the regulating clock, delayed excitation on an ultrasonic transducer once or multiple times; a data collection module configured to perform data collection on an ultrasonic echo signal of the ultrasonic transducer and signals after each time of performing delayed excitation; a data synthesis module configured to synthesize and superimpose data as collected to obtain high sampling rate data; and an ultrasonic imaging module configured to perform ultrasonic imaging according to the high sampling rate data.

Preferably, the delayed excitation module is further configured to take one period of the regulating clock as a delay period, and perform delayed excitation on the ultrasonic transducer once according to the delay period.

Preferably, the delayed excitation module is further configured to take one period of the regulating clock as a delay period, and perform delayed excitation on the ultrasonic transducer once according to this delay period; take two periods of the regulating clock as a delay period, and perform delayed excitation on the ultrasonic transducer once according to this delay period; in a similar fashion, take multiple periods of the regulating clock as a delay period, and perform delayed excitation on the ultrasonic transducer once according to this delay period.

Preferably, a first collection unit is configured to perform data collection on the ultrasonic echo signal at a rising edge of a sampling clock; and a second collection unit is configured to perform data collection on signals after each time of performing delayed excitation at a rising edge of the sampling clock.

Preferably, the data synthesis module is further configured to synthesize and superimpose the data as collected in accordance with a time-sequential relationship of delayed excitation to obtain the high sampling rate data.

Preferably, the ultrasonic transducer is a single ultrasonic transducer, or an ultrasonic array transducer consisting of a plurality of ultrasonic transducer elements.

According to a further aspect of the invention, there is provided with a delayed excitation system, wherein the delayed excitation system includes: a signal receiving module configured to receive an ultrasonic signal and convert the ultrasonic signal as received into an ultrasonic echo signal; a programmable logic control module configured to generate a regulating clock and transmit a control signal of a delayed excitation to a signal transmitting module, and further configured to collect data of a delayed excitation, and synthesize and superimpose the data as collected to obtain high sampling rate data; a signal transmitting module configured to perform a delayed excitation on the ultrasonic echo signal according to the regulating clock after receiving the control signal transmitted by the programmable logic control module.

Preferably, the signal receiving module includes: a switch configured to be turned off when the ultrasonic signal is transmitted and turned on when the ultrasonic signal is received; a low noise signal amplification unit (LNA) configured to perform a first level of amplification on the ultrasonic signal as received; a programmable gain amplification unit (PGA) configured to amplify the ultrasonic signal after the first level of amplification once more, wherein an amplification factor is adjusted by the programmable logic control module; a low pass filter (LPF) configurable analog filtering unit configured to adjust a cut-off frequency of low-pass filtering, and filter out high-frequency noise having a frequency higher than a cut-off frequency in the ultrasonic signal that are amplified by the programmable gain amplification circuit (PGA); a high precision A/D conversion unit (ADC) configured to convert the ultrasonic signal filtered out by the LPF configurable analog filtering unit into the ultrasonic echo signal.

Preferably, the programmable logic control module includes: a serial peripheral interface (SPI) logic control unit configured to control the signal transmitting module; a delayed excitation regulating clock unit configured to generate a regulating clock; a sampling data synthesis unit configured to collect the data of delayed excitation, and synthesize and superimpose the data as collected to obtain the high sampling rate data.

Preferably, the signal transmitting module includes: a metal-oxide-semiconductor field effect transistor (MOSFET) driver configured to receive the control signal of the programmable logic control module, amplify the control signal and then transmit it to a MOSFET high voltage conduction unit; the MOSFET high voltage conduction unit configured to perform a delayed excitation on the ultrasonic echo signal according to the control signal as received; and an impedance matching high voltage excitation unit configured to match different types of ultrasonic transducers.

The invention provides a delayed excitation ultrasonic imaging method and apparatus, and a delayed excitation system. By performing delayed excitation on the ultrasonic transducer, a conventional analog-to-digital conversion chip can also perform high-frequency ultrasonic imaging, and a solution of performing ultrasonic imaging by low-cost sampling and delayed excitation can be implemented. The present invention can be used not only in a traditional low-frequency ultrasonic imaging system, but also in a high-frequency ultrasonic imaging system, which can greatly reduce the system cost.

### BRIEF DESCRIPTION OF DRAWINGS

The drawings described herein are used for providing further understanding to the present invention and constitute a part of the present application, and schematic embodiments of the present invention and the description thereof are used for explaining the present invention and are not intended to limit the present invention. In the drawings:

FIG. 1 is a flowchart of a delayed excitation ultrasonic imaging method according to an embodiment of the present invention;

FIG. 2 is a schematic diagram of performing delayed excitation on the ultrasonic transducer once according to an embodiment of the present invention;

FIG. 3 is a first schematic diagram of data synthesis according to an embodiment of the present invention;

FIG. 4 is a schematic diagram of performing delayed excitation on the ultrasonic transducer multiple times according to an embodiment of the present invention;

FIG. 5 is a second schematic diagram of data synthesis according to an embodiment of the present invention;

FIG. 6 is a structural schematic diagram of a delayed excitation ultrasonic imaging apparatus according to an embodiment of the present invention;

FIG. 7 is a structural schematic diagram of a data collection module according to an embodiment of the present invention;

FIG. 8 is a structural schematic diagram of a delayed excitation system according to an embodiment of the present invention;

FIG. 9 is an overall block diagram of the delayed excitation system according to an embodiment of the present invention;

FIG. 10 is a structural schematic diagram of a signal receiving module according to an embodiment of the present invention;

FIG. 11 is a structural schematic diagram of a programmable logic control module according to an embodiment of the present invention;

FIG. 12 is a schematic diagram of an SPI logic control unit according to an embodiment of the present invention;

FIG. 13 is a structural schematic diagram of a signal transmitting module according to an embodiment of the present invention;

FIG. 14 is a schematic diagram of data processing results of the measured signal according to an embodiment of the present invention;

FIG. 15 is a schematic diagram of data processing results of a signal after delayed excitation according to an embodiment of the present invention;

FIG. 16 is a schematic diagram of data superimposing results according to an embodiment of the present invention;

FIG. 17 is a schematic diagram of data synthesis processing results according to an embodiment of the present invention.

### DESCRIPTION OF EMBODIMENTS

Hereinafter the technical solution in the embodiments of the present invention will be described clearly and completely in combination with the accompanying drawings in the embodiments of the present invention, and obviously, the described embodiments are merely part of the embodiments, not all of the embodiments. Based on the embodiments of the present invention, all other embodiments that are obtained by persons skilled in the art without paying creative efforts fall within the protection scope of the present invention.

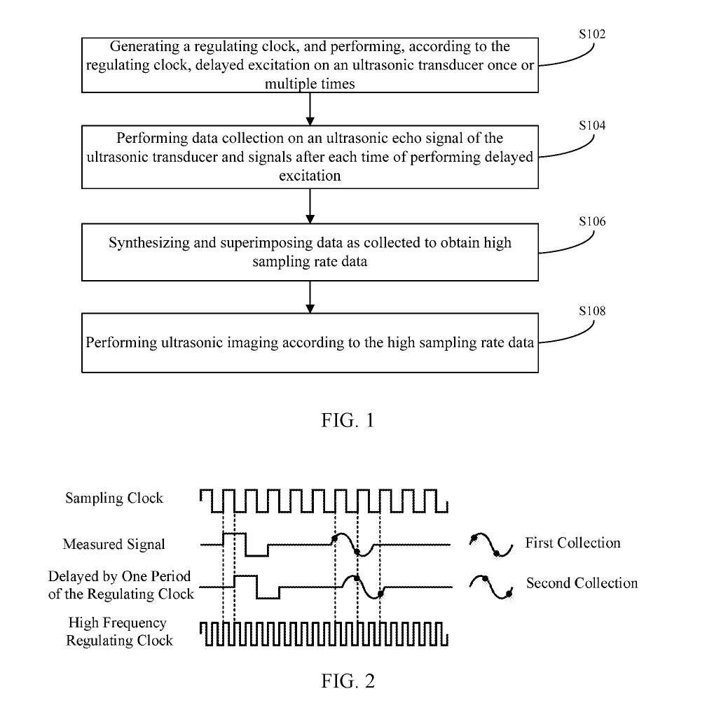

An embodiment of the present invention provides a delayed excitation ultrasonic imaging method, FIG. 1 is a flowchart of the delayed excitation ultrasonic imaging method according to an embodiment of the present invention, as shown in FIG. 1, the method includes the following steps (steps S102-S108):

step S102: generating a regulating clock, and performing, according to the regulating clock, delayed excitation on an ultrasonic transducer once or multiple times;

step S104: performing data collection on an ultrasonic echo signal of the ultrasonic transducer and signals after each time of performing delayed excitation;

step S106: synthesizing and superimposing data as collected to obtain high sampling rate data; and

step S108: performing ultrasonic imaging according to the high sampling rate data.

By performing delayed excitation on the ultrasonic transducer, the embodiment of the present invention enables a conventional analog-to-digital conversion chip also to perform high-frequency ultrasonic imaging, and a solution of performing ultrasonic imaging by low-cost sampling and delayed excitation can be implemented. The technical solution provided by the embodiment can be used not only in a traditional low-frequency ultrasonic imaging system but also in a high-frequency ultrasonic imaging system, which can greatly reduce the system cost.

In the present embodiment, the number of times of performing delayed excitation on the ultrasonic transducer can be one or multiple, which can be set according to specific demands. After delayed excitation, it needs to perform data collection on the ultrasonic echo signal at a rising edge of the sampling clock; and to perform data collection on signals after each time of performing delayed excitation at a rising edge of the sampling clock. Then, the data as collected are synthesized and superimposed in accordance with a time-sequential relationship of delayed excitation to obtain high sampling rate data. The more the number of times of performing delayed excitation is, the higher the precision of the obtained high sampling rate data will be.

If it is set that delayed excitation is performed on the ultrasonic transducer once, then one period of the regulating clock is taken as a delay period, and delayed excitation is performed on the ultrasonic transducer once according to the delay period. FIG. 2 is a schematic diagram of performing delayed excitation on the ultrasonic transducer once according to an embodiment of the present invention. As shown in FIG. 2, a high-frequency regulating clock is used for regulating delay, in FIG. 2, the time of delaying one adjustment clock cycle is taken as an example, the delayed measured signal (an ultrasonic echo signal) as shown in FIG. 2 may be staggered by one phase and then collected again at a clock rising edge of ADC.

The data collection after delayed excitation is performed twice: the first collection is to obtain two sampling points at the rising edge of the sampling clock; at the time of the second collection, because the ultrasonic excitation is performed for the time delayed by one regulating clock, the ultrasonic echo signal also arrives late by one period of the regulating clock and is collected at the time staggered by one phase, thus two sampling points are obtained at the rising edge of the sampling clock.

FIG. 3 is a first schematic diagram of data synthesis according to an embodiment of the present invention. As shown in FIG. 3, only two sampling points of the measured signal can be collected at a sampling frequency of the measured signal, this is not enough for data analysis and not enough to satisfy Nyquist's law, so delayed excitation is performed once, and the delay time is one period of a high-frequency regulating clock, correspondingly to the rising edge of the sampling clock, and thus two sampling points with staggered phases are collected, then the sampling points of the measured signals and the sampling points after the delayed excitation are synthesized and superimposed according to a time-sequential relationship of delayed excitation by logic programming, to obtain high sampling rate data.

Of course, FIG. 2 makes explanations only by taking the time delayed by one period of the regulating clock as an example, the time of two, three or four periods of the regulating clock can also be set flexibly as needed, and more times of sampling can be performed depending on different number of times of delay, thereby a better sampling result can be obtained.

If it is set that delayed excitation is performed on the ultrasonic transducer multiple times, then: one period of the regulating clock is taken as a delay period, and delayed excitation is performed once on the ultrasonic transducer according to this delay period; two periods of the regulating clock are taken as a delay period, and delayed excitation is performed once on the ultrasonic transducer according to this delay period; in a similar fashion, multiple periods of the regulating clock are taken as a delay period, and delayed excitation is performed once on the ultrasonic transducer according to this delay period.

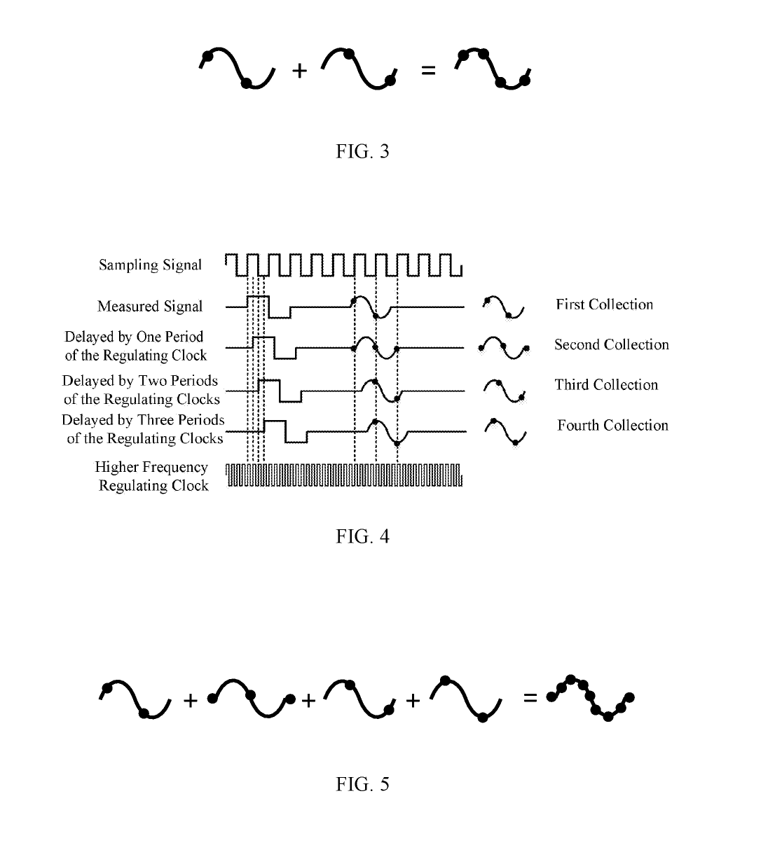

FIG. 4 is a schematic diagram of performing delayed excitation on the ultrasonic transducer multiple times according to an embodiment of the invention. As shown in FIG. 4, data collection is performed on the measured signal (ultrasonic echo signal) at the rising edge of the sampling clock, then data collection is performed in sequence on the measured signals delayed by one period of the regulating clock, two periods of the regulating clock and three periods of the regulating clock.

FIG. 5 is a second schematic diagram of data synthesis according to the embodiment of the present invention. As shown in FIG. 5, because delayed excitation is performed on the ultrasonic transducer three times in FIG. 4, correspondingly data may be collected for four times. The four sets of data are synthesized and superimposed to obtain the high sampling rate data.

FIG. 4 and FIG. 5 show processes of three times of performing delayed excitation and four times of data collection, there are eight sampling points after a plurality of times of synthesis of the sampling data, which is four times of that of a conventional manner. The number of times of performing delayed excitation can be expanded flexibly and applied as needed. In this embodiment, three, four or more sets of data are synthesized by three, four or more times of performing delayed excitation. If two sets of data are synthesized, two times of the sampling precision can be obtained. The more the number of times of performing delayed excitation is, the sampling precision is also correspondingly promoted in multiples.

In this embodiment, the ultrasonic transducer can be a single ultrasonic transducer, or can be an ultrasonic array transducer consisting of a plurality of ultrasonic transducer elements. It is not limited in the present invention.

Based on the same inventive concept, an embodiment of the present invention further provides a delayed excitation ultrasonic imaging apparatus which can be used for implementing the method described in the above embodiment, as described in the embodiment below. Since the principle based on which the delayed excitation ultrasonic imaging apparatus solves the problem is similar to that of the delayed excitation ultrasonic imaging method, therefore, the implementation of the delayed excitation ultrasonic imaging apparatus can refer to the implementation of the delayed excitation ultrasonic imaging method, and is not repeated here. As used below, the term “unit” or “module” is a combination of software and/or hardware that can implement a predetermined function. Although preferably the system described in the following embodiment is implemented by software, implementation by hardware, or a combination of software and hardware is also possible and is conceivable.

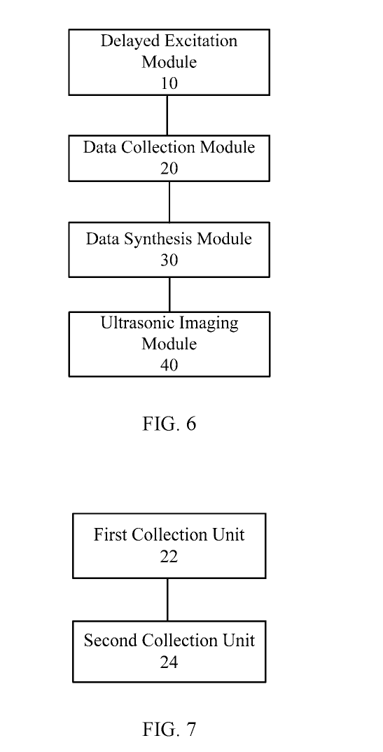

FIG. 6 is a structural schematic diagram of a delayed excitation ultrasonic imaging apparatus according to an embodiment of the invention. As shown in FIG. 6, the apparatus includes: a delayed excitation module 10, a data collection module 20, a data synthesis module 30 and an ultrasonic imaging module 40, and the structure is described in detail as below.

The delayed excitation module 10 is configured to generate a regulating clock, and perform, according to the regulating clock, delayed excitation on an ultrasonic transducer once or multiple times.

The data collection module 20 is configured to perform data collection on an ultrasonic echo signal of the ultrasonic transducer and signals after each time of performing delayed excitation.

The data synthesis module 30 is configured to synthesize and superimpose the data as collected to obtain high sampling rate data.

The ultrasonic imaging module 40 is configured to perform ultrasonic imaging according to the high sampling rate data.

By performing delayed excitation on the ultrasonic transducer, the embodiment of the present invention enables a conventional analog-to-digital conversion chip to perform high-frequency ultrasonic imaging, and a solution of performing ultrasonic imaging by low-cost sampling and delayed excitation can be implemented. The technical solution provided by the present embodiment can be used not only in a traditional low-frequency ultrasonic imaging system, but also in a high-frequency ultrasonic imaging system, which can greatly reduce the system cost.

The number of times that the ultrasonic transducer performs delayed excitation can be one or multiple, which can be set according to specific demands. In the present embodiment, the delayed excitation module 10 is configured to take one period of the regulating clock as a delay period, and perform delayed excitation on the ultrasonic transducer once according to the delay period. The delayed excitation module 10 is further configured to take one period of the regulating clock as a delay period, and perform delayed excitation on the ultrasonic transducer once according to this delay period; take two periods of the regulating clock as a delay period, and perform delayed excitation on the ultrasonic transducer once according to this delay period; in a similar fashion, take multiple periods of the regulating clock as a delay period, and perform delayed excitation on the ultrasonic transducer once according to this delay period. The more the number of times of performing delayed excitation is, the higher the precision of the obtained high sampling rate data will be.

FIG. 7 is a structural schematic diagram of a data collection module according to an embodiment of the present invention. As shown in FIG. 7, the data collection module 20 includes: a first collection unit 22 configured to perform data collection on the ultrasonic echo signal at a rising edge of the sampling clock; and a second collection unit 24 configured to perform data collection on signals after each time of performing delayed excitation at a rising edge of the sampling clock. The number of times of data collection is generally one more time than that of performing delayed excitation. The more the number of times of the data collection is, the higher the sampling precision is.

In one embodiment, the data synthesis module 30 is configured to synthesize and superimpose the data as collected in accordance with a time-sequential relationship of delayed excitation to obtain high sampling rate data. Thus, the high sampling rate data with high accuracy and high precision can be obtained.

In this embodiment, the ultrasonic transducer can be a single ultrasonic transducer, or can be an ultrasonic array transducer consisting of a plurality of ultrasonic transducer elements. It is not limited in the present invention.

Of course, the above-described module division is merely a schematic division, and the present invention is not limited to this. The apparatus can also only include: a delayed excitation module, a data processing module and an ultrasonic imaging module, wherein the delayed excitation module performs functions related to delayed excitation, the data processing module performs functions related to data collection and data synthesis, and the ultrasonic imaging module performs a function related to ultrasonic imaging. Any module division should fall within the protection scope of the present invention as long as it can realize the purpose of the present invention.

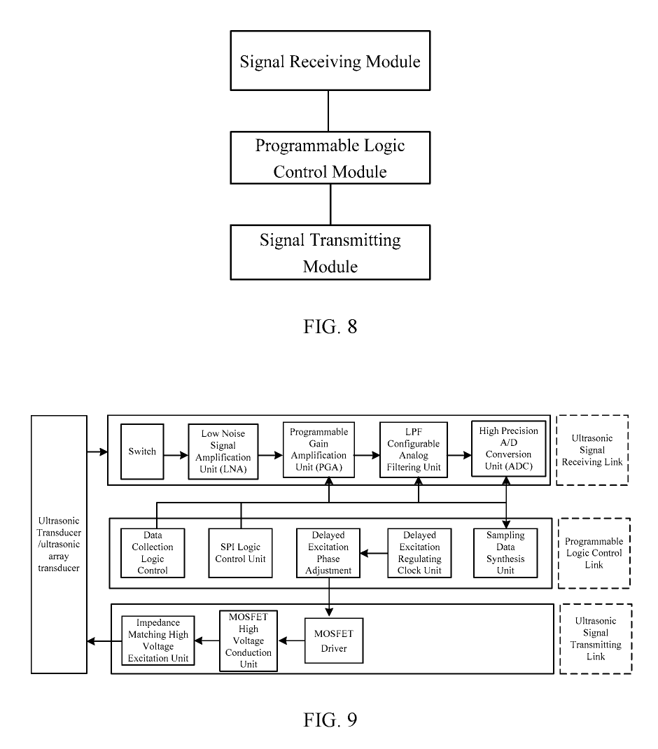

Based on the same inventive concept, an embodiment of the present invention further provides a delayed excitation system which can be used for implementing the method described in the above embodiment. FIG. 8 is a structural schematic diagram of a delayed excitation system according to an embodiment of the present invention. As shown in FIG. 8, the system includes: a signal receiving module, a programmable logic control module and a signal transmitting module, and its structure is described in detail as below.

The signal receiving module is configured to receive an ultrasonic signal and then convert the ultrasonic signal into an ultrasonic echo signal.

The programmable logic control module is configured to generate a regulating clock to transmit a control signal of delayed excitation to the signal transmitting module, and is further configured to collect data of delayed excitation, and synthesize and superimpose the data as collected to obtain high sampling rate data.

The signal transmitting module is configured to perform delayed excitation on the ultrasonic echo signal according to the regulating clock after receiving the control signal transmitted by the programmable logic control module.

By performing delayed excitation on the ultrasonic transducer, this embodiment enables a conventional analog-to-digital conversion chip also to perform high-frequency ultrasonic imaging, and a solution of performing ultrasonic imaging by low-cost sampling and delayed excitation can be implemented. The technical solution provided by this embodiment can be used not only in a traditional low-frequency ultrasonic imaging system, but also in a high-frequency ultrasonic imaging system, which can greatly reduce the system cost.

FIG. 9 is an overall block diagram of the delayed excitation system according to an embodiment of the present invention. As shown in FIG. 9, the delayed excitation system is divided mainly into three links, i.e., three parts of ultrasonic signal reception, programmable logic control and ultrasonic signal transmission. In FIG. 9, each unit can be implemented independently by using one functional circuit, and each module can be implemented by integrating with one integrated chip.

The technical solution of the present application can be applied to a single ultrasonic transducer, or can also be applied to an array transducer consisting of a plurality of ultrasonic transducer elements, the ultrasonic transducer/the array transducer plays the role of receiving or transmitting an ultrasonic signal.

(I) An Ultrasonic Signal Receiving Link

After receiving an ultrasonic signal, the ultrasonic transducer or the array transducer converts the acoustic signal into an electric signal, the electric signal passes through a switch to enter a data collection channel, at this time the electric signal is very weak, and a low noise signal amplification needs to be performed on the electric signal by a low noise amplifier (LNA), and then the electric signal is amplified again by a programmable gain amplifier (PGA), passes through a Low Pass Filter (LPF) configurable analog filtering module and then enters an analog-to-digital converter (ADC) to convert an analog signal into a digital signal.

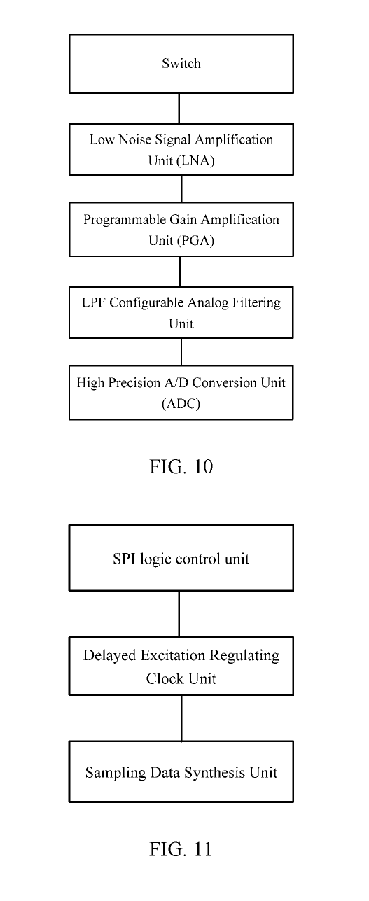

The signal receiving module can adopt an integrated dedicated chip, and can also set up separate analog circuits. FIG. 10 is a structural schematic diagram of a signal receiving module according to an embodiment of the invention. As shown in FIG. 10, the signal receiving module includes:

a switch that is turned off when an ultrasonic signal is transmitted and is turned on when an ultrasonic signal is received, the transmitting/receiving switch mainly playing a role of protecting a receiving circuit, and can prevent high voltage excitations of the transmitting circuit from flowing to the receiving circuit, thereby damaging the electronic devices;

a low noise signal amplification unit (LNA) for configured to perform a first level of amplification on the ultrasonic signal as received. It is possible to introduce less other noise signals on the basis of maximizing the amplification of the ultrasonic signals;

a programmable gain amplification unit (PGA) configured to amplify the ultrasonic signal after the first level of amplification once more; wherein the amplification factor is adjusted by the programmable logic control module. The gain (an amplified value) of the programmable gain amplification unit (PGA) amplified by the previous level LNA is often insufficient and it needs to be amplified once more to obtain a satisfactory amplification effect. In the present invention, a flexible design is made, that is, the programmable gain amplification unit (PGA) can be controlled by a core logic device field programmable gate array (FPGA) by an SPI serial communication bus to realize that the gain is adjustable, i.e., the amplification factor can be adjusted by programming;

an LPF configurable analog filtering unit configured to adjust the cut-off frequency of low-pass filtering, to filter out high-frequency noise having a frequency higher than the cut-off frequency in the ultrasonic signals that are amplified by the programmable gain amplification unit (PGA). The LPF configurable analog filtering unit can be a configurable analog low pass filter, can be controlled by the core logic device FPGA by the SPI serial communication bus, can adjust the cut-off frequency of low-pass filtering according to a change of signals, to filter out the high-frequency noise having a frequency higher than the cut-off frequency;

a high precision A/D conversion unit (ADC) configured to convert the ultrasonic signal filtered out by the LPF configurable analog filtering unit into an ultrasonic echo signal. The analog signal that has subjected to a previous level of amplification and low-pass filtering can enter the high precision A/D conversion unit (ADC) to perform data collection. The setting of the working parameters of the high precision A/D conversion unit (ADC) can also be controlled by the core logic device FPGA by the SPI serial communication bus, and the sampling frequency and the sampling precision can be adjusted according to the change of signals.

(II) A Programmable Logic Control Link

This link works in the programmable logic device FPGA, is responsible for global logic control, includes: working time sequence of the ultrasonic signal receiving link, working time sequence of the ultrasonic signal transmitting link, and generation of a high-frequency regulating clock within the programmable logic device FPGA to perform control of delayed excitation, and synthesizing of sampling results of delayed excitation to improve an effective sampling rate.

The programmable logic control module is designed and completed on FPGA by programming, or can also be replaced with a solution of building a digital circuit or a complex programmable logic device (CPLD) to a certain extent. FIG. 11 is a structural schematic diagram of a programmable logic control module according to an embodiment of the present invention. As shown in FIG. 11, the programmable logic control module includes:

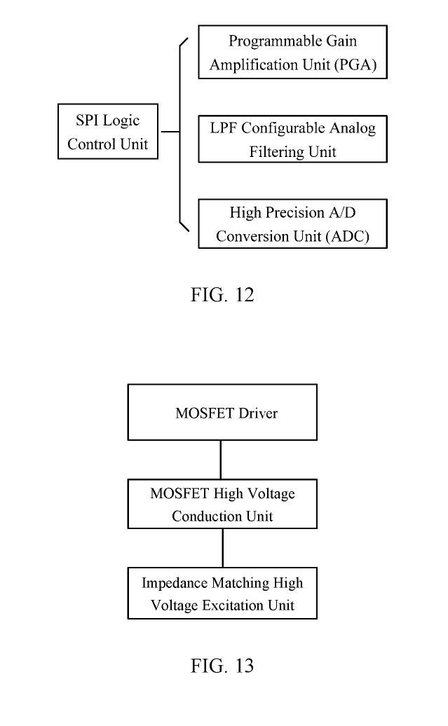

an SPI logic control unit configured to control a signal transmitting module. In the present invention, a circuit design and function matching are performed based on its actual needs, the FPGA is programmed by hardware description language, an SPI logic control module within the FPGA is designed, thereby functional reconstruction of a conventional chip being completed. FIG. 12 is a schematic diagram of an SPI logic control unit according to an embodiment of the present invention. As shown in FIG. 12, the SPI logic control unit controls other three units, including a programmable gain amplification unit (PGA), an LPF configurable analog filtering unit and a high precision A/D conversion unit (ADC). Meanwhile, a data collection logic control module within the FPGA also programs the FPGA through hardware description languages, which coordinates the time sequence problem of the entire data collection process, drives an ADC chip to work, and etc. The FPGA can be understood as a chip that can realize logic functions by programming, which has a very high operating efficiency and also saves the board space and the hardware cost, and when it is necessary to modify the functions, it is needed to rewrite the program to download, and there is no need to replace the device, which is very flexible.

A delayed excitation regulating clock unit is configured to generate a regulating clock. It is assumed that the working frequency of the ultrasonic transducer adopted in the present invention is 40 MHz, the sampling frequency of the analog-to-digital conversion chip as adopted is 80 MHz, and there is a key design point here, i.e., a higher speed regulating clock of 160 Mz is generated within the programmable logic device. Because the higher speed regulating clock has a finer phase adjustment capability, which provides a necessary condition for the delayed excitation phase adjustment of a latter level. The sampling clock is a work clock of the analog-to-digital conversion chip (ADC), and the data collection and conversion are performed at the time of a rising edge of the sampling clock. In FIG. 2 and FIG. 4, in the measured signals, the square wave in the front is a high voltage excitation for the ultrasonic transducer and is just for illustration and can be ignored. What is important is the subsequent ultrasonic echo signal in sinusoidal wave shape which is an object to be collected, and an echo frequency is equivalent to an excitation frequency.

A sampling data synthesis unit is configured to collect the data of delayed excitation, and synthesize and superimpose the data as collected to obtain the high sampling rate data.

(III) An Ultrasonic Signal Transmitting Link

In this link, after having received the control signal transmitted from a front-end programmable logic device, a high voltage excitation is performed on the ultrasonic transducer or the array transducer by a MOSFET driver and a MOSFET high voltage conduction unit.

The signal transmitting module can adopt an integrated dedicated chip, and may also build a discrete analog circuit. FIG. 13 is a structural schematic diagram of a signal transmitting module according to an embodiment of the invention. As shown in FIG. 13, the signal transmitting module includes:

a MOSFET driver configured to receive a control signal of the programmable logic control module, amplify the control signal and then transmit it to a MOSFET high voltage conduction unit. The MOSFET driver is controlled by the front-end programmable logic device, and its function is that because the programmable logic device cannot directly drive a metal-oxide-semiconductor field effect transistor (MOSFET), there is a need to provide a MOSFET driving circuit to receive the control signal of the programmable logic device, amplify the control signal here and then drive a subsequent level of MOSFET high voltage conduction unit. The change of the control signal lies only in amplification of voltage and current, the phase relationship and shape of the control signal are not changed.

A MOSFET high voltage conduction unit is configured to perform delayed excitation on the ultrasonic echo signal according to the received control signal. The MOSFET high voltage conduction unit is controlled by a previous level of MOSFET driver to conduct positive high voltage and negative high voltage according to a change of the control signal, to form high voltage excitation on the ultrasonic transducer.

An impedance matching high voltage excitation unit mainly consists of a transformer and other discrete elements and is used for matching different types of ultrasonic transducers.

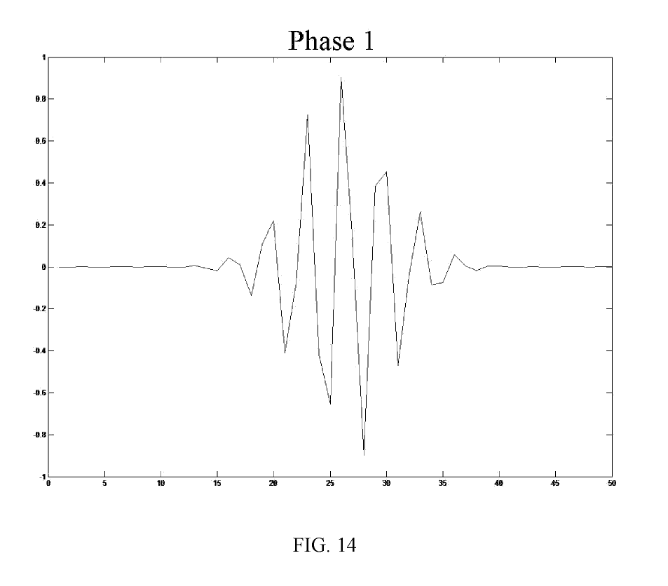

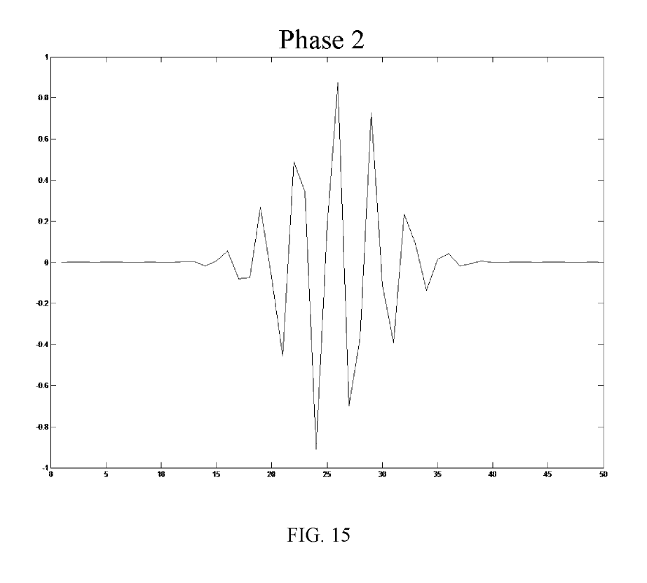

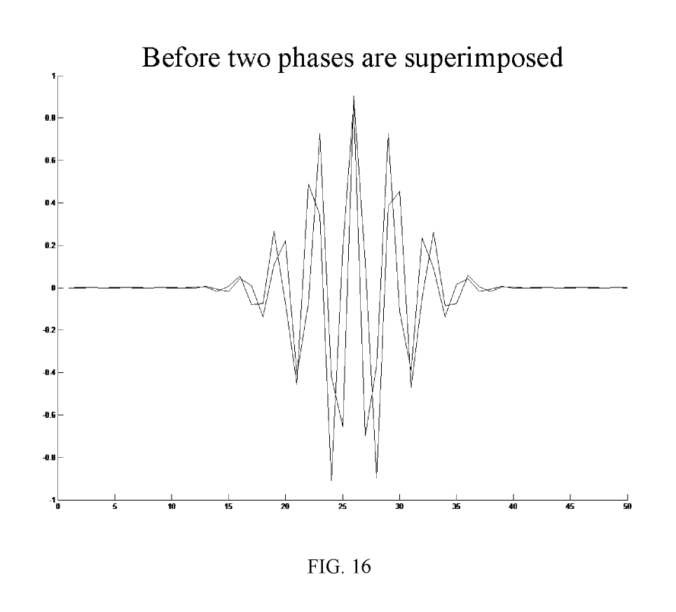

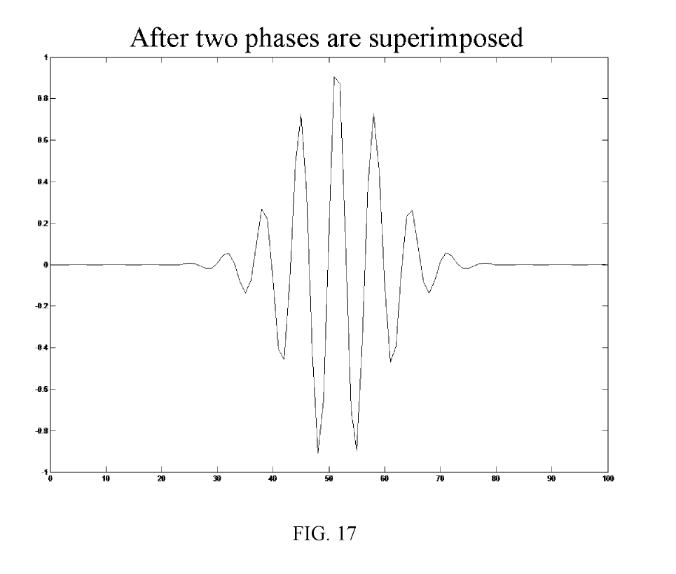

FIG. 14 is a schematic diagram of a data processing result of the measured signal according to an embodiment of the present invention. FIG. 15 is a schematic diagram of a data processing result of a signal after delayed excitation according to an embodiment of the present invention. FIG. 16 is a schematic diagram of a result of data superimposing according to an embodiment of the present invention. FIG. 17 is a schematic diagram of a data synthesis processing result according to an embodiment of the present invention. A waveform line of the measured signal shown in FIG. 14 is coarse. FIG. 15 shows a waveform of the signal after delayed excitation is performed once on the ultrasonic transducer. The signal data in FIG. 14 and FIG. 15 are superimposed to obtain the waveform shown in FIG. 16 and then data synthesis is performed thereon to obtain a waveform as shown in FIG. 17 having a line being smooth and fine.

It can be seen from FIGS. 14 to 17 that, the waveform shape is not good when it serves as independent data because the sampling rate is not enough, but after algorithm synthesis, the superimposed image is more reflective of the real situation in the waveform form, because the number of sampling points is doubled, the waveform is complete and free of distortion.

High frequency ultrasonic imaging technology is getting more and more attention and expectation from the medical community because it can obtain clearer medical diagnostic images on special diagnostic occasions to help doctors analyze a patient's condition. The high frequency ultrasonic imaging technology also becomes increasingly important at the forefront of science because the scientific research of many small animals and humans can be made, and it is an indispensable scientific research tool. In the present invention, by generating a high-frequency regulating clock, the delayed excitation is performed on the ultrasonic transducer, to realize ultrasonic imaging by multiple collections and data synthesis. The invention can be applied in traditional low-frequency ultrasonic imaging field, which can greatly reduce the system cost.

In the description, the descriptions with reference to the terms “one embodiment”, “some embodiments”, “example”, “specific example” or “some examples” and the like mean that the specific features, structures, materials or characteristics described in combination with the embodiment or example are included in at least one embodiment or example of the present invention. In the description, exemplary expressions of the above terms do not necessarily refer to the same embodiment or example. Moreover, the described specific features, structures, materials or characteristics may be combined in a suitable manner in any one or more of the embodiments or examples.

The objects, technical solutions and beneficial effects of the present invention have been further described in detail in the above specific embodiments, it should be understood that the above contents are merely specific embodiments of the present invention and are not for limiting the protection scope of the present invention, and any modification, equivalent replacement, improvement and the like within the spirit and principle of the present invention shall fall within the protection scope of the present invention.

What is claimed is:

1. A delayed excitation ultrasonic imaging method, comprising:
generating a regulating clock, and performing, according to the regulating clock, delayed excitation on an ultrasonic transducer once or multiple times;
performing data collection on an ultrasonic echo signal of the ultrasonic transducer and signals after each time of performing delayed excitation;
synthesizing and superimposing data as collected to obtain high sampling rate data; and
performing ultrasonic imaging according to the high sampling rate data.

2. The delayed excitation ultrasonic imaging method according to claim 1, wherein the step of performing, according to the regulating clock, delayed excitation on the ultrasonic transducer once comprises:
taking one period of the regulating clock as a delay period, and performing delayed excitation on the ultrasonic transducer once according to the delay period.

3. The delayed excitation ultrasonic imaging method according to claim 1, wherein the step of performing, according to the regulating clock, delayed excitation on the ultrasonic transducer multiple times comprises:
taking one period of the regulating clock as a delay period, and performing delayed excitation on the ultrasonic transducer once according to this delay period;
taking two periods of the regulating clock as a delay period, and performing delayed excitation on the ultrasonic transducer once according to this delay period;
in a similar fashion,
taking multiple periods of the regulating clock as a delay period, and performing delayed excitation on the ultrasonic transducer once according to this delay period.

4. The delayed excitation ultrasonic imaging method according to claim 1, wherein the step of performing data collection on the ultrasonic echo signal of the ultrasonic transducer and the signals after each time of performing delayed excitation comprises:
performing data collection on the ultrasonic echo signal at a rising edge of a sampling clock; and
performing data collection on the signals after each time of performing delayed excitation at a rising edge of the sampling clock.

5. The delayed excitation ultrasonic imaging method according to claim 1, wherein the step of synthesizing and superimposing the data as collected to obtain the high sampling rate data comprises:
synthesizing and superimposing the data as collected in accordance with a time-sequential relationship of delayed excitation to obtain the high sampling rate data.

6. The delayed excitation ultrasonic imaging method according to claim 1, wherein the ultrasonic transducer is a single ultrasonic transducer, or an ultrasonic array transducer consisting of a plurality of ultrasonic transducer elements.

7. A delayed excitation ultrasonic imaging apparatus, comprising:
a delayed excitation module configured to generate a regulating clock, and perform, according to the regulating clock, delayed excitation on an ultrasonic transducer once or multiple times;
a data collection module configured to perform data collection on an ultrasonic echo signal of the ultrasonic transducer and signals after each time of performing delayed excitation;
a data synthesis module configured to synthesize and superimpose data as collected to obtain high sampling rate data; and
an ultrasonic imaging module configured to perform ultrasonic imaging according to the high sampling rate data.

8. The delayed excitation ultrasonic imaging apparatus according to claim 7, wherein the delayed excitation module is further configured to take one period of the regulating clock as a delay period, and perform delayed excitation on the ultrasonic transducer once according to the delay period.

9. The delayed excitation ultrasonic imaging apparatus according to claim 7, wherein the delayed excitation module is further configured to: take one period of the regulating clock as a delay period, and perform delayed excitation on the ultrasonic transducer once according to this delay period; take two periods of the regulating clock as a delay period, and perform delayed excitation on the ultrasonic transducer once according to this delay period; in a similar fashion, take multiple periods of the regulating clock as a delay period, and perform delayed excitation on the ultrasonic transducer once according to this delay period.

10. The delayed excitation ultrasonic imaging apparatus according to claim 7, wherein the data collection module comprises:
a first collection unit configured to perform data collection on the ultrasonic echo signal at a rising edge of a sampling clock; and
a second collection unit configured to perform data collection on signals after each time of performing delayed excitation at a rising edge of the sampling clock.

11. The delayed excitation ultrasonic imaging apparatus according to claim 7, wherein the data synthesis module is further configured to synthesize and superimpose the data as collected in accordance with a time-sequential relationship of delayed excitation to obtain the high sampling rate data.

12. The delayed excitation ultrasonic imaging apparatus according to claim 7, wherein the ultrasonic transducer is a single ultrasonic transducer, or an ultrasonic array transducer consisting of a plurality of ultrasonic transducer elements.

13. A delayed excitation system, comprising:
a signal receiving module configured to receive an ultrasonic signal and convert the ultrasonic signal as received into an ultrasonic echo signal;
a programmable logic control module configured to generate a regulating clock and transmit a control signal of delayed excitation to a signal transmitting module, and further configured to collect data of delayed excitation, and synthesize and superimpose the data as collected to obtain high sampling rate data; and
a signal transmitting module configured to perform delayed excitation on the ultrasonic echo signal according to the regulating clock after receiving the control signal transmitted by the programmable logic control module.

14. The delayed excitation system according to claim 13, wherein the signal receiving module comprises:
a switch configured to be turned off when the ultrasonic signal is transmitted and turned on when the ultrasonic signal is received;
a low noise signal amplification unit (LNA) configured to perform a first level of amplification on the ultrasonic signal as received;
a programmable gain amplification unit (PGA) configured to amplify the ultrasonic signal after the first level of amplification once more, wherein an amplification factor is adjusted by the programmable logic control module;
a low pass filter (LPF) configurable analog filtering unit configured to adjust a cut-off frequency of low-pass filtering, and filter out high-frequency noise having a frequency higher than a cut-off frequency in the ultrasonic signal that is amplified by the programmable gain amplification unit (PGA); and
a high precision A/D conversion unit (ADC) configured to convert the ultrasonic signal filtered out by the LPF configurable analog filtering unit into the ultrasonic echo signal.

15. The delayed excitation system according to claim 13, wherein the programmable logic control module comprises:
a serial peripheral interface (SPI) logic control unit configured to control the signal transmitting module;
a delayed excitation regulating clock unit configured to generate a regulating clock; and
a sampling data synthesis unit configured to collect the data of delayed excitation, and synthesize and superimpose the data as collected to obtain the high sampling rate data.

16. The delayed excitation system according to claim 13, wherein the signal transmitting module comprises:
a metal-oxide-semiconductor field effect transistor (MOSFET) driver configured to receive the control signal of the programmable logic control module, amplify the control signal and then transmit it to a MOSFET high voltage conduction unit;
the MOSFET high voltage conduction unit configured to perform delayed excitation on the ultrasonic echo signal according to the control signal as received; and
an impedance matching high voltage excitation unit configured to match different types of ultrasonic transducers.
